(19) United States
(12) Patent Application Publication
(10) Pub. No.: US 2002/0161309 A1
(43) Pub. Date: Oct. 31, 2002

(54) Fiber optic power source for an electroencephalograph acquisition apparatus

(75) Inventor: Dominic P. Marro, North Andover, MA (US)

Correspondence Address: Burns & Levinson LLP, Suite 300, 1030 Fifteenth Street N.W., Washington, DC, 20005-1501, US

(73) Assignee: Physiometrix, Inc., Billerica, MA

(21) Appl. No.: 10/183,752

(22) Filed: Jun. 26, 2002

Related U.S. Application Data
(62) Division of application No. 09/699,122, filed on Oct. 27, 2000.
(60) Provisional application No. 60/161,644, filed on Oct. 27, 1999.

(51) Int. Cl. A61B 5/04

(52) U.S. Cl. 600/544

(57) ABSTRACT

A reusable appliance for acquisition from a patient of EEG signals comprising a micro-miniature, micro-power, low noise multi-channel data-acquisition system powered by a fiber optic illuminator and photovoltaic cell thus eliminating the need for inductively coupled power converters. The appliance also uses disposable EEG electrodes which are similar in size and very low cost.

This application claims the benefit of priority under co-pending United States Provisional Application No. 60/161,644, filed Oct. 27, 1999.

### FIELD OF THE INVENTION

The current invention relates to the field of medical anesthesia. More particularly it relates to the field of electronic monitoring of a patient undergoing anesthesia, especially for use during and after surgical operations. The invention more specifically relates to the device used to acquire electrical electroencephalograph (EEG) signals used to monitor a patient's state of awareness, more specifically still to the device comprising and obtaining electroencephalograph signals from one or more electrodes attached to the patient's head.

### BACKGROUND OF THE INVENTION

Traditionally in the administration of anesthesia it has been the practice for an anesthesiologist to use only clinical signs from the patient to estimate the depth of the patient's anesthesia before and during surgical procedures requiring anesthesia. In recent years, however, it has become possible and practicable to manipulate certain transduced bodily signals, in particular electro-encephalographic (EEG) signals, to produce an indication of how anesthetized or alternatively how awake a patient is. The raw EEG signals are acquired via gel or other conducting electrodes attached to one or more predetermined standard locations on the patient's head. These electrodes are typically used in the operating room (OR) or in the intensive care unit (ICU).

A system for electronically monitoring a patient's state of anesthesia using electroencephalograph signals is described in a prior patent application, Ennen, et al., U. S. patent application Ser. No. 09/431,632, filed Nov. 2, 1999, which is incorporated by reference herein. That application describes, among other things, the signals which will be gathered from the patient's head and as well the information which will be produced for the anesthesia practitioner but does not disclose the most effective way of acquiring the raw signals from the patient's head.

Signal acquisition for patient monitoring use in the OR and ICU presents a number of design challenges that can and will effect the overall acceptance of the product based on its cost, ease of use and performance. A typical prior art design costs approximately eight dollars per unit to manufacture and has 7 leads that need to be affixed to the patient's head prior to commencing the surgical procedure. Anterior (frontal), central and posterior electrode sites in this array monitor both hairy and hairless areas of the scalp requiring the use of different electrode types and tensioning elements for reliable contact. Although designed for “ease of use,” a 7 lead device is inherently more difficult to use and expensive than, for example, a 3-lead frontal array. Other inventors have developed such arrays and have produced disposable versions thereof. Nevertheless, such disposable attachments cost on the order of two dollars to manufacture

In addition, the harsh EMI environment of the operating room challenges a monitoring system, since electrosurgical devices produce signals up to 1 billion times greater in amplitude than that of the EEG. In order to minimize the coupling of these undesired signals to the EEG sensors and leadwires, shielded leadwires and short sensor leads are standard practice.

There are situations, however, where electrocautery is in close proximity to the PSA preamplifier and signal corruption occurs. When this does happen, it either increases latency of a displayed index during artifact recognition and rejection, or increases signal variability and index magnitude when not recognized as artifact. Another source of noise/artifact is caused by leadwire movement. This gives rise to the triboelectric effect, and other low frequency friction-charge effects. This type of artifact will also increase latency of the displayed index during artifact recognition and rejection and an increase in signal variability with a decrease in the computed index when not recognized.

There has also been an increase in the use of MRI combined with other physiological monitoring modalities. Transformer isolated power supplies are useless in the presence of the high fields near an MRI as their cores become saturated rendering them totally in-effective. In addition the, the leakage fields associated with these compromised inductors and transformers interferes with the sensitive measurements performed during MRI data acquisition.

There is a need for a patient acquisition subsystem which on the one hand has very low consumables costs and on the other hand provides resistance to external electromagnetic signals and reduces interference with diagnostic equipment such as MRI scanners. It is therefore an object of the current invention to produce a patient interface with very low per use cost and significantly improved artifact rejection to both high frequency, intense signals such as electrocautery while eliminating most low frequency noise associated with either patient or patient lead movement. An additional object of this invention is to eliminate the bulky preamplifier and incorporate the necessary components for signal acquisition, patient protection, shielding and filtering into a small reusable element. It is a further object of this invention to provide a device in which disposable, that is, consumable, elements, are as inexpensive as possible. An additional object of this invention is to provide the ability to monitor patients in the presence of the intense fields associated with the MRI or MRT. An additional object of this invention is to provide a means for remote monitoring in harsh environments where it is desirable to have the highest degree of electrical isolation for remote sensing for improved noise, safety and economy. Such applications include, but are not limited to aircraft fuel monitoring systems, oil and gas exploration and chemical plant process control monitoring.

### SUMMARY OF THE INVENTION

The reusable appliance of this invention comprises a micro-miniature, micropower, low noise multi-channel data-acquisition system powered by a fiber optic illuminator and photovoltaic cell, thus eliminating the need for inductively coupled power converters. The appliance uses disposable EEG electrodes which are similar in size and have an estimated cost to produce of approximately $0.02 each.

### BRIEF DESCRIPTION OF THE DRAWINGS

FIG. 1 portrays the basic system configuration of the signal acquisition module.

FIG. 2 is a diagram of the data acquisition system

FIG. 3 shows the configuration of the fiber optic power system as well as features of the data acquisition system.

FIG. 4 shows a typical disposable gel electrode with conductive adhesive substrate.

### DESCRIPTION OF THE PREFERRED EMBODIMENT

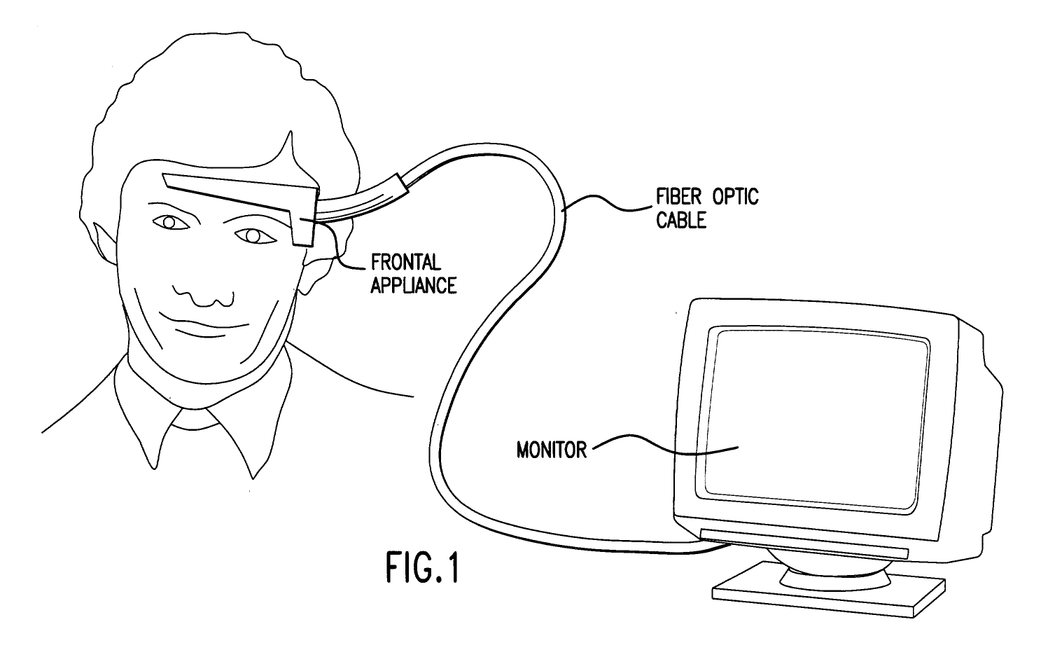

As shown in FIG. 1, the electronics at the patient end of the cable is housed in a semi-rigid enclosure. Internal circuitry is wrapped in a flexible conductive Faraday shield layer connected to the amplifier's signal ground. The circuit layout is designed such that the contact patch for each of the signal electrodes is incorporated at the intended recording site for the Frontal Array signal montage. These patches are the only portions of the electronics external to the shield layer.

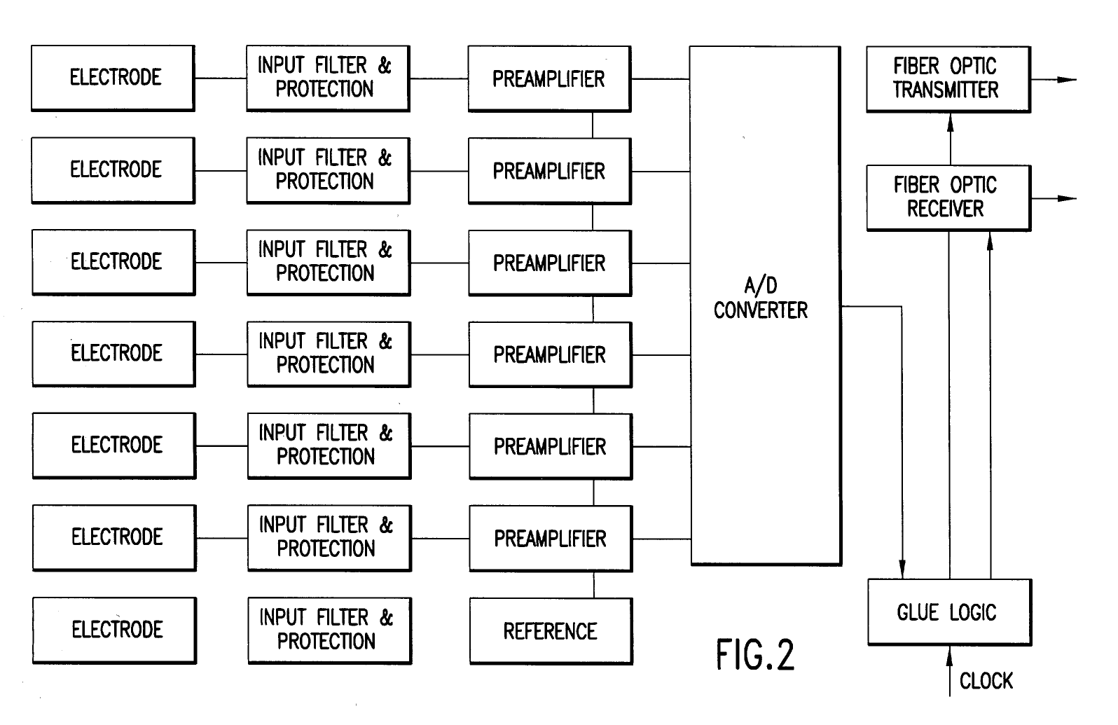

A system diagram is shown in FIG. 2. Each of the signals is filtered and voltage limited through the unique application of a voltage limiter/capacitor at each of the amplifier's signal inputs. This device is the first leg of a multistage R-C-R-C filter optimized for the EEG signal bandwidth. This device also provides protection to the patient and EEG amplifier meeting IEC601-2-26 “Particular requirements for the safety of electroencephalographs” when used with defibrillators in accordance with the “Rationale for Defibrillator Test Voltages”. The use of this device, a Transorb, manufactured by AVX, is unique in that the manufacturer does not specify this device for the ultra-low leakage use with EEG amplifiers. A thorough review of the manufacturer's specifications and analysis of an idealized model of this part reveals that the leakage current levels, when used with low differential offset (<10 millivolt) Physiometrix or equivalent electrodes, are unmeasurable.

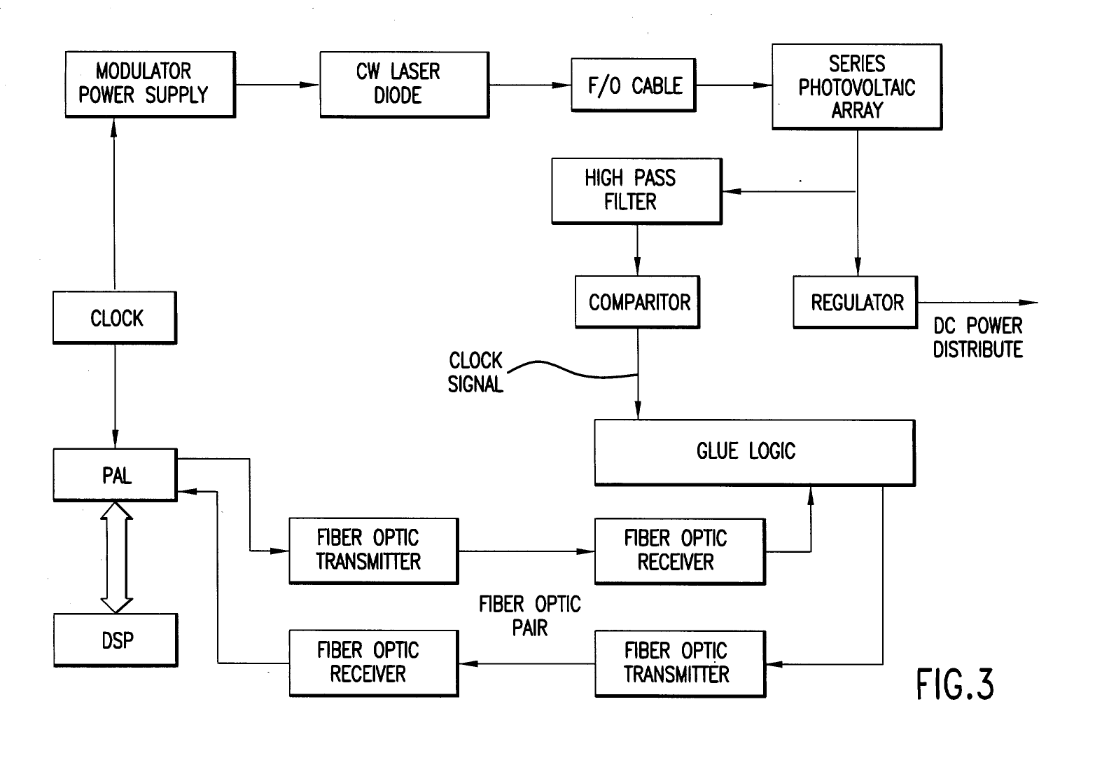

A diagram encompassing the fiber optic power generation system along with a schematic of the data transmission system is shown in FIG. 3. The fiber optic cable provides total isolation from coupling capacitance associated with transformer based isolated power supplies. A power source, such as Opto Power H01-A001-mmm-Ct/100, comprises a CW diode laser coupled to a standard 100-μm core F/O cable. Any appropriately matched F/O cable may be used. The remote end of the cable is attached to a re-usable appliance, which houses a multi-channel, micro-power data acquisition system. Optical power is converted back to electrical power using several series connected photovoltaic cells coupled to the output of the F/O cable. The optical interface to the F/O cable is unique, in that it is optimized for uniformly spreading, optical power over several photovoltaic cells, producing an unregulated voltage greater than 5.0 volts. With typical conversion efficiencies of 15%, 6 dB of cable transmission losses for a 5 meter link and coupling losses between the F/O cable, up to 100 milliwatts of power is available. Incorporating the AID clock with the optical power source further reduces power requirements and circuit complexity for the remote electronics. This is accomplished by modulating the laser diode at the intended A/D clock frequency and recovering the clock at the remote end of the cable. Ripple from the modulated optical beam will be present at the output of the photocells. AC coupling of this signal, prior to filtering and regulation, to an analog comparitor will provide a recovered clock signal suitable for proper operation of the A/D converter. Other signals, such as the chip select and data conversion strobe are recovered from the serial data stream. A linear regulator is used to provide 5.0 volts for the amplifier, A/D converter, glue logic and optical communications transceiver. Power requirements for the electronics is approximately 50 milliwatts. Two or more optical fibers attached to LEDs and Phototransistors such as HPXXXX and HPXXXX at both ends of a compatible fiber optic cable provide full duplex data-communications.

The data communication and power cables are incorporated into a single multifiber cable. The remotely powered electronics includes micro-power amplifiers, such as, the Analog Devices OPX97 family or Linear Technology LT1114 with a six channel LTC1293 or equivalent data acquisition system. The multi-channel EEG data is transmitted to the DSP via a F/O link. The data are reformatted using programmable array logic (PAL) and input to the DSP's serial I/O port for filtering and decimation. The electronics defined herein facilitates amplifier calibration on start-up and continuous electrode impedance monitoring.

Electrode sites may include any hairy or hairless site on the scalp. This appliance and electrode design, however, is optimized to use three or more of the following 10-20 defined sites. FpZ, Fp1, Fp2, F3, F4, F7, F8, M1 and or M2. Contact pad geometry facilitates the use of electrode arrays optimized for different head sizes. Rapid attachment of the disposable electrodes is facilitated through the use of a double-sided conduction layer and or conductive snaps incorporated into the Frontal Electrodes or Frontal Electrode Array. The Frontal Array, which is defined in greater detail in a separate application, United States Provisional Application No. 60/213,642, filed Jun. 23, 2000, utilizes adhesive rings surrounding the Ag, AgCl conductive gel sensor to secure the appliance to the patient's head.

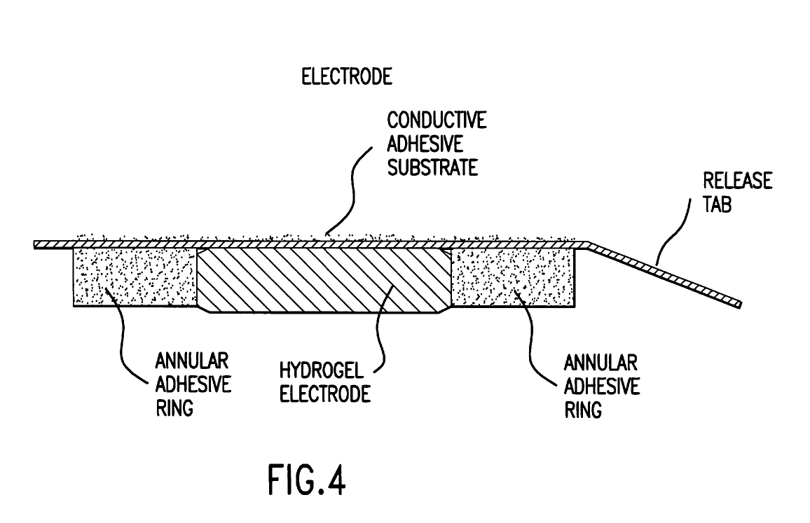

A typical gel electrode is shown in FIG. 4. The electrode unit is configured with conducting material on one surface. A disposable gel electrode suitable for use with the instant invention has been described in a United States patent application by the inventor herein. In particular, Marro, U.S. Application Ser. No. 09/431,966, filed Nov. 1, 1999, discloses reservoir electrodes of a type which could be used with the instant invention. Other electrodes of a disposable nature, such as EKG gel electrodes, could also be used.

I claim

1. A reusable apparatus for the acquisition of electroencephalographic signals from the head of a patient, the apparatus comprising a patient appliance having an array comprising a plurality of low cost disposable EEG electrodes, a power system comprising a fiber optic illuminator and photovoltaic cell, whereby the need for inductively coupled power converters is eliminated, and an optical data transmission cable.

2. The apparatus of claim 1 in which the optical data transmission cable and the fiber optic illuminator comprise a single optical fiber cable.

3. The apparatus of claim 2 in which the patient appliance and the single optical fiber cable are substantially enclosed in a Faraday shield electrically connected ground.

4. A fiber optic power system for low interference remote acquisition of electrical signals comprising a power source, a diode laser, a fiber optic cable, an optical interface,and a plurality of photovoltaic cells.

5. The fiber optic power system of claim 4 in which the optical interface is configured to spread optical power substantially evenly over the plurality of photovoltaic cells.

6. The fiber optic power system of claim 4 additionally comprising an analog to digital clock.

7. The apparatus of claim 1 in which the patient appliance further comprises a micro-miniature, micro-power, low noise multi-channel data-acquisition system.

8. The apparatus of claim 7 in which the a micro-miniature, micro-power, low noise multi-channel data-acquisition system comprises remotely powered electronics comprising micro-power amplifiers.
